(19) United States
(12) Patent Application Publication
(10) Pub. No.: US 2009/0326572 A1
(43) Pub. Date: Dec. 31, 2009

(54) APPARATUS AND METHODS FOR RAPID TISSUE CROSSING

(76) Inventors: Ruey-Feng PEH, Mountain View, CA (US); David MILLER, Cupertino, CA (US); Zachary J. MALCHANO, San Francisco, CA (US); Vahid SAADAT, Atherton, CA (US)

Correspondence Address: LEVINE BAGADE HAN LLP, 2400 GENG ROAD, SUITE 120, PALO ALTO, CA, 94303, US

(21) Appl. No.: 12/483,119

(22) Filed: Jun. 11, 2009

Related U.S. Application Data
(60) Provisional application No. 61/076,514, filed on Jun. 27, 2008.

(51) Int. Cl. A61M 29/00

(52) U.S. Cl. 606/192

(57) ABSTRACT

Apparatus and methods for rapid tissue crossing are described utilizing a device to penetrate and rapidly cross a tissue layer in a patient body without the need to withdraw the tissue visualization catheter out of the patient body to be replaced with a separate dilator. The distal end of a dilator sheath, within which the visualization device is positionable, may be collapsible to form a conical dilator. Upon the placement of the tip of the dilator at the site of transseptal puncture, the conical dilator may be advanced distally through the puncture to enlarge the opening. With passage of the dilator sheath through the opening, a visualization hood may be advanced and deployed through the conical dilator which opens from its conical shape to allow the passage of the hood or other instruments therethrough.

### CROSS-REFERENCE TO RELATED APPLICATIONS

This application claims the benefit of priority to U.S. Prov. Pat. App. 61/076,514 filed Jun. 27, 2008, which is incorporated herein by reference in its entirety.

### FIELD OF THE INVENTION

The present invention relates generally to medical devices used for rapidly crossing through a tissue region. More particularly, the present invention relates to apparatus and methods for facilitating the rapid crossing of intravascular instruments through tissue regions such as an inter-atrial septum for transseptal procedures.

### BACKGROUND OF THE INVENTION

Conventional devices for visualizing interior regions of a body lumen are known. For example, ultrasound devices have been used to produce images from within a body in vivo. Ultrasound has been used both with and without contrast agents, which typically enhance ultrasound-derived images.

Other conventional methods have utilized catheters or probes having position sensors deployed within the body lumen, such as the interior of a cardiac chamber. These types of positional sensors are typically used to determine the movement of a cardiac tissue surface or the electrical activity within the cardiac tissue. When a sufficient number of points have been sampled by the sensors, a “map” of the cardiac tissue may be generated.

Another conventional device utilizes an inflatable balloon which is typically introduced intravascularly in a deflated state and then inflated against the tissue region to be examined. Imaging is typically accomplished by an optical fiber or other apparatus such as electronic chips for viewing the tissue through the membrane(s) of the inflated balloon. Moreover, the balloon must generally be inflated for imaging. Other conventional balloons utilize a cavity or depression formed at a distal end of the inflated balloon. This cavity or depression is pressed against the tissue to be examined and is flushed with a clear fluid to provide a clear pathway through the blood.

However, such imaging balloons have many inherent disadvantages. For instance, such balloons generally require that the balloon be inflated to a relatively large size which may undesirably displace surrounding tissue and interfere with fine positioning of the imaging system against the tissue. Moreover, the working area created by such inflatable balloons are generally cramped and limited in size. Furthermore, inflated balloons may be susceptible to pressure changes in the surrounding fluid. For example, if the environment surrounding the inflated balloon undergoes pressure changes, e.g., during systolic and diastolic pressure cycles in a beating heart, the constant pressure change may affect the inflated balloon volume and its positioning to produce unsteady or undesirable conditions for optimal tissue imaging.

Moreover, such visualization devices and methods may present difficulties when utilized for traversing through a tissue region, such as passing transseptally through a tissue wall. Transseptal tissue procedures typically require the use of multiple instruments such as piercing needles and tissue dilation tools. This necessitates multiple insertions and withdrawals of several instruments and generally increases the risk to patients. Moreover, such procedures are performed without the benefit of direct visualization of the underlying tissue to be pierced and traversed, additionally raising the risk to the patient.

Thus, there is a need for a device which is configured to provide direct visualization of tissue while also providing for the rapid crossing of the tissue region such as for gaining transseptal access.

### BRIEF SUMMARY OF THE INVENTION

A tissue imaging and manipulation apparatus that may be utilized for procedures within a body lumen, such as the heart, in which visualization of the surrounding tissue is made difficult, if not impossible, by medium contained within the lumen such as blood, is described below. Generally, such a tissue imaging and manipulation apparatus comprises an optional delivery catheter or sheath through which a deployment catheter and imaging hood may be advanced for placement against or adjacent to the tissue to be imaged.

The deployment catheter may define a fluid delivery lumen therethrough as well as an imaging lumen within which an optical imaging fiber or assembly may be disposed for imaging tissue. When deployed, the imaging hood may be expanded into any number of shapes, e.g., cylindrical, conical as shown, semi-spherical, etc., provided that an open area or field is defined by the imaging hood. The open area is the area within which the tissue region of interest may be imaged. The imaging hood may also define an atraumatic contact lip or edge for placement or abutment against the tissue region of interest. Moreover, the distal end of the deployment catheter or separate manipulatable catheters may be articulated through various controlling mechanisms such as push-pull wires manually or via computer control

The deployment catheter may also be stabilized relative to the tissue surface through various methods. For instance, inflatable stabilizing balloons positioned along a length of the catheter may be utilized, or tissue engagement anchors may be passed through or along the deployment catheter for temporary engagement of the underlying tissue.

In operation, after the imaging hood has been deployed, fluid may be pumped at a positive pressure through the fluid delivery lumen until the fluid fills the open area completely and displaces any blood from within the open area. The fluid may comprise any biocompatible fluid, e.g., saline, water, plasma, Fluorinert™, etc., which is sufficiently transparent to allow for relatively undistorted visualization through the fluid. The fluid may be pumped continuously or intermittently to allow for image capture by an optional processor which may be in communication with the assembly.

In an exemplary variation for imaging tissue surfaces within a heart chamber containing blood, the tissue imaging and treatment system may generally comprise a catheter body having a lumen defined therethrough, a visualization element disposed adjacent the catheter body, the visualization element having a field of view, a transparent fluid source in fluid communication with the lumen, and a barrier or membrane extendable from the catheter body to localize, between the visualization element and the field of view, displacement of blood by transparent fluid that flows from the lumen, and an instrument translatable through the displaced blood for performing any number of treatments upon the tissue surface within the field of view. The imaging hood may be formed into any number of configurations and the imaging assembly may also be utilized with any number of therapeutic tools which may be deployed through the deployment catheter.

More particularly in certain variations, the tissue visualization system may comprise components including the imaging hood, where the hood may further include a membrane having a main aperture and additional optional openings disposed over the distal end of the hood. An introducer sheath or the deployment catheter upon which the imaging hood is disposed may further comprise a steerable segment made of multiple adjacent links which are pivotably connected to one another and which may be articulated within a single plane or multiple planes. The deployment catheter itself may be comprised of a multiple lumen extrusion, such as a four-lumen catheter extrusion, which is reinforced with braided stainless steel fibers to provide structural support. The proximal end of the catheter may be coupled to a handle for manipulation and articulation of the system.

To provide visualization, an imaging element such as a fiberscope or electronic imager such as a solid state camera, e.g., CCD or CMOS, may be mounted, e.g., on a shape memory wire, and positioned within or along the hood interior. A fluid reservoir and/or pump (e.g., syringe, pressurized intravenous bag, etc.) may be fluidly coupled to the proximal end of the catheter to hold the translucent fluid such as saline or contrast medium as well as for providing the pressure to inject the fluid into the imaging hood.

Generally, for the visualization and treatment devices to traverse through a punctured tissue wall, the opening through the tissue wall is typically dilated prior to passage. This may typically require several withdrawals and exchanges of various instruments such as a dilator or ablation device to widen the tissue opening to allow for the atraumatic passage of instruments such as the visualization and treatment device.

However, one example of a device which may allow for the penetration and rapid crossing of a tissue wall utilizing a dilation sheath. Such a dilation sheath may have a flexible length through which the visualization assembly may be advanced and a dilation assembly positioned along a distal end of the sheath which may enable the penetration and rapid crossing of a septal wall without the use of a separate dilator and also without having to withdraw the visualization and treatment device from the patient body to allow for introduction of a separate dilator and the subsequent reinsertion of the visualization device.

The hood may project from the deployment catheter into its deployed configuration for positioning against the septal wall, which may be imaged to visually confirm a location of the hood, e.g., along the fossa ovalis. The sheath may be optionally deployed as well for conveying the hood and catheter. With the hood placed against the tissue surface and visual confirmation of the tissue location obtained, a piercing needle may be advanced through the catheter and hood to pierce into and through the atrial septum while under visualization from the imager. A guidewire may be introduced through the needle and also through the formed tissue opening such that the guidewire passes from the right atrium to the left atrium.

With the guidewire passing through the opening, the visualization assembly may be withdrawn directly within the dilation sheath or optionally within the sheath. As the sheath, or the visualization device itself, is further withdrawn within the dilation sheath or as dilation sheath is advanced over the sheath or the visualization device, the dilation assembly may be biased to collapse or reconfigure itself into a tapered dilation configuration. The dilation assembly may be comprised, in one example, of a covering or extension which is formed of several triangular or saw-tooth shaped portions interconnected by an elastomeric substance in an alternating pattern along biased portions and attached to dilation sheath at attachment. As the sheath or visualization device is withdrawn and dilation assembly is unconstrained, the elastomeric portions may be biased to draw each individual portion towards one another to collapse the assembly while forming a guidewire opening through which the guidewire may pass. Alternatively, the dilation assembly may be formed of a single construct such as a distensible membrane or covering which is biased to collapse when a constraint is removed.

In either case, once the dilation assembly has been reconfigured into its tapered configuration, the dilation sheath with the visualization device positioned within may be advanced along the guidewire and through the tissue opening until the dilation assembly is positioned distal to the opening within, e.g., left atrium. The tapered configuration of the dilation assembly may accordingly facilitate the dilation and passage of the dilation sheath through the atrial septum. Once the dilation assembly has passed desirably through the septal wall, the sheath (or the visualization device itself) may be advanced relative to the dilation sheath such that the dilation assembly is expanded back to its opened configuration to allow for the catheter and hood to be deployed again while in the left atrium where it may be advanced into proximity to any region of tissue for visualization and/or treatment such as the ostial tissue around the pulmonary veins, electrophysiological signal mapping, visualization, ablation, or other therapeutic and diagnostic procedures.

In yet another example, hood may incorporate an inflatable dilator balloon which is optionally integrated along a distal end of the hood and expanded to form a conical dilator which projects distally of the hood while maintaining a guidewire opening. Alternatively, a deflated conically-shaped dilation balloon may be advanced from one of the working channels of the catheter and inflated within the visualization hood into a conical shape. In either variation, the inflated conical dilation balloon may be subsequently deployed and pushed distally along the guidewire to enlarge the transseptal puncture to dilate the opening while the hood remains in its deployed configuration.

In yet another variation, the visualization device may be utilized with an inflatable balloon dilator. After introduction of the guidewire into the left atrium, a dilation balloon shaft having a dilation balloon may be advanced over the guidewire in an uninflated state and into the undilated tissue opening. When desirably positioned within opening, the dilation balloon may be inflated to expand the opening to a diameter which is at least as wide as catheter, if not wider, or optionally as wide as the deployed hood.

While maintaining the dilation balloon in its inflated state, the hood may be collapsed and withdrawn within the sheath and the sheath may then be advanced relative to the balloon so that the distal opening of the sheath is just proximal to the inflated balloon. Once the sheath with the collapsed hood has gained access into the left atrium, the hood may be readily redeployed from the sheath and the dilation balloon may be deflated and subsequently withdrawn through the hood and the catheter.

Yet another variation may comprise a hood assembly covered by a membrane and which defines an aperture over the membrane at a distal end of the hood. The hood may be defined by several support struts which extend from the proximal end of the hood and define curved or bent portions which terminate at the distal end of the hood at the flow control aperture. To deploy and/or collapse the hood between its deployed and low-profile configurations, an instrument such as a dilator having an atraumatic tip projecting distally from a shoulder may be advanced distally through the deployment catheter and into the hood. In particular, the atraumatic tip may be electrically coupled to a power source, such as an RF generator, such that the tip is energizable with RF energy and functions as a bipolar or monopolar energizable cutting electrode.

The instrument may be further advanced until the tip projects through the aperture and the shoulder engages or abuts against the interior of the membrane surrounding the aperture. As the instrument is pushed further distally, the curved or bent portions of the support struts may become start to become straightened relative to the instrument and the support struts may begin to collapse. In addition to the tip, all or selected numbers of the support struts may be coupled to a power source, such as the same RF generator coupled to the tip, and may have exposed portions which are energizable to facilitate dilation or cutting of the tissue opening to facilitate the passage of the collapsed hood.

With the hood in its low-profile configuration with the tip extended distally, RF energy may be applied to the tip when in contact with or in proximity to the tissue region to be crossed. As the energized tip begins to cut through the tissue, the hood may be urged distally through the tissue opening. One or more struts may be optionally energized to facilitate the cutting and dilation of the opening as the hood is urged further through the opening. Once the streamlined low-profile hood is fully positioned within the left atrium, the tip may then be withdrawn proximally to restore the hood back into its deployed configuration.

### BRIEF DESCRIPTION OF THE DRAWINGS

FIG. 1A shows a side view of one variation of a tissue imaging apparatus during deployment from a sheath or delivery catheter.

FIG. 1B shows the deployed tissue imaging apparatus of FIG. 1A having an optionally expandable hood or sheath attached to an imaging and/or diagnostic catheter.

FIG. 1C shows an end view of a deployed imaging apparatus.

FIGS. 2A and 2B show one example of a deployed tissue imager positioned against or adjacent to the tissue to be imaged and a flow of fluid, such as saline, displacing blood from within the expandable hood.

FIGS. 3A and 3B show examples of various visualization imagers which may be utilized within or along the imaging hood.

FIGS. 4A and 4B show perspective and end views, respectively, of an imaging hood having at least one layer of a transparent elastomeric membrane over the distal opening of the hood.

FIGS. 5A and 5B show perspective and end views, respectively, of an imaging hood which includes a membrane with an aperture defined therethrough and a plurality of additional openings defined over the membrane surrounding the aperture.

FIGS. 6A to 6E illustrate one example of a dilation sheath which has a dilation assembly which is reconfigurable from an open configuration through which a visualization device may be advanced to a tapered dilation configuration which may be advanced through a tissue opening to dilate the opening and convey the visualization device through the opening.

FIGS. 7A to 7D illustrate another example of a tissue dilation device which may incorporate an inflatable dilator balloon optionally integrated along a distal end of the hood and expandable to form a conical dilator.

FIGS. 8A to 8D illustrate another example of a tissue dilation device utilizing an inflatable dilator balloon which is inflatable distal to the collapsed hood and advanced through a tissue opening along with the visualization device positioned proximally.

FIGS. 9A to 9D illustrate another example of a tissue dilation device where the hood is distally collapsible via an instrument having an energizable tip extending distal to the collapsed hood and one or more energizable struts.

FIGS. 10A to 10C illustrate an example of the device of FIGS. 9A to 9D advanced through an atrial tissue wall while in a low-profile configuration with an energized tip and/or struts forming an opening through the tissue.

### DETAILED DESCRIPTION OF THE INVENTION

A tissue-imaging and manipulation apparatus described herein is able to provide real-time images in vivo of tissue regions within a body lumen such as a heart, which is filled with blood flowing dynamically therethrough and is also able to provide intravascular tools and instruments for performing various procedures upon the imaged tissue regions. Such an apparatus may be utilized for many procedures, e.g., facilitating transseptal access to the left atrium, cannulating the coronary sinus, diagnosis of valve regurgitation/stenosis, valvuloplasty, atrial appendage closure, arrhythmogenic focus ablation, among other procedures.

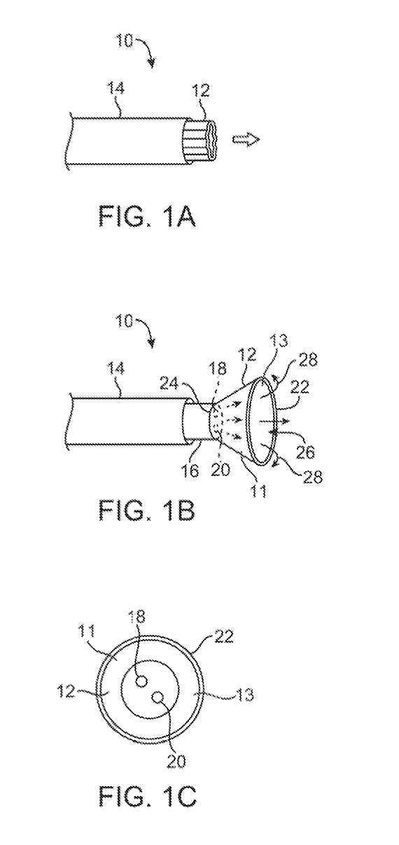

One variation of a tissue access and imaging apparatus is shown in the detail perspective views of FIGS. 1A to 1C. As shown in FIG. 1A, tissue imaging and manipulation assembly 10 may be delivered intravascularly through the patient's body in a low-profile configuration via a delivery catheter or sheath 14. In the case of treating tissue, it is generally desirable to enter or access the left atrium while minimizing trauma to the patient. To non-operatively effect such access, one conventional approach involves puncturing the intra-atrial septum from the right atrial chamber to the left atrial chamber in a procedure commonly called a transseptal procedure or septostomy. For procedures such as percutaneous valve repair and replacement, transseptal access to the left atrial chamber of the heart may allow for larger devices to be introduced into the venous system than can generally be introduced percutaneously into the arterial system.

When the imaging and manipulation assembly 10 is ready to be utilized for imaging tissue, imaging hood 12 may be advanced relative to catheter 14 and deployed from a distal opening of catheter 14, as shown by the arrow. Upon deployment, imaging hood 12 may be unconstrained to expand or open into a deployed imaging configuration, as shown in FIG. 1B. Imaging hood 12 may be fabricated from a variety of pliable or conformable biocompatible material including but not limited to, e.g., polymeric, plastic, or woven materials. One example of a woven material is Kevlar® (E. I. du Pont de Nemours, Wilmington, Del.), which is an aramid and which can be made into thin, e.g., less than 0.001 in., materials which maintain enough integrity for such applications described herein. Moreover, the imaging hood 12 may be fabricated from a translucent or opaque material and in a variety of different colors to optimize or attenuate any reflected lighting from surrounding fluids or structures, i.e., anatomical or mechanical structures or instruments. In either case, imaging hood 12 may be fabricated into a uniform structure or a scaffold-supported structure, in which case a scaffold made of a shape memory alloy, such as Nitinol, or a spring steel, or plastic, etc., may be fabricated and covered with the polymeric, plastic, or woven material. Hence, imaging hood 12 may comprise any of a wide variety of barriers or membrane structures, as may generally be used to localize displacement of blood or the like from a selected volume of a body lumen or heart chamber. In exemplary embodiments, a volume within an inner surface 13 of imaging hood 12 will be significantly less than a volume of the hood 12 between inner surface 13 and outer surface 11.

Imaging hood 12 may be attached at interface 24 to a deployment catheter 16 which may be translated independently of deployment catheter or sheath 14. Attachment of interface 24 may be accomplished through any number of conventional methods. Deployment catheter 16 may define a fluid delivery lumen 18 as well as an imaging lumen 20 within which an optical imaging fiber or assembly may be disposed for imaging tissue. When deployed, imaging hood 12 may expand into any number of shapes, e.g., cylindrical, conical as shown, semi-spherical, etc., provided that an open area or field 26 is defined by imaging hood 12. The open area 26 is the area within which the tissue region of interest may be imaged. Imaging hood 12 may also define an atraumatic contact lip or edge 22 for placement or abutment against the tissue region of interest. Moreover, the diameter of imaging hood 12 at its maximum fully deployed diameter, e.g., at contact lip or edge 22, is typically greater relative to a diameter of the deployment catheter 16 (although a diameter of contact lip or edge 22 may be made to have a smaller or equal diameter of deployment catheter 16). For instance, the contact edge diameter may range anywhere from 1 to 5 times (or even greater, as practicable) a diameter of deployment catheter 16. FIG. 1C shows an end view of the imaging hood 12 in its deployed configuration. Also shown are the contact lip or edge 22 and fluid delivery lumen 18 and imaging lumen 20.

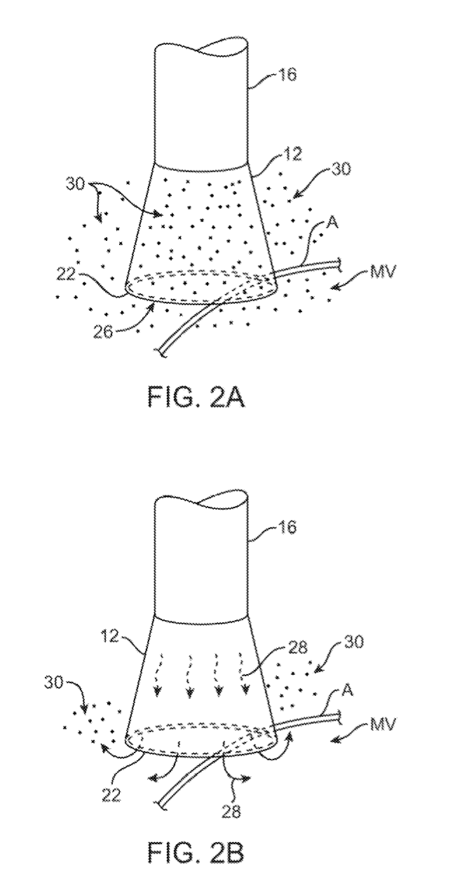

As seen in the example of FIGS. 2A and 2B, deployment catheter 16 may be manipulated to position deployed imaging hood 12 against or near the underlying tissue region of interest to be imaged, in this example a portion of annulus A of mitral valve MV within the left atrial chamber. As the surrounding blood 30 flows around imaging hood 12 and within open area 26 defined within imaging hood 12, as seen in FIG. 2A, the underlying annulus A is obstructed by the opaque blood 30 and is difficult to view through the imaging lumen 20. The translucent fluid 28, such as saline, may then be pumped through fluid delivery lumen 18, intermittently or continuously, until the blood 30 is at least partially, and preferably completely, displaced from within open area 26 by fluid 28, as shown in FIG. 2B.

Although contact edge 22 need not directly contact the underlying tissue, it is at least preferably brought into close proximity to the tissue such that the flow of clear fluid 28 from open area 26 may be maintained to inhibit significant backflow of blood 30 back into open area 26. Contact edge 22 may also be made of a soft elastomeric material such as certain soft grades of silicone or polyurethane, as typically known, to help contact edge 22 conform to an uneven or rough underlying anatomical tissue surface. Once the blood 30 has been displaced from imaging hood 12, an image may then be viewed of the underlying tissue through the clear fluid 30. This image may then be recorded or available for real-time viewing for performing a therapeutic procedure. The positive flow of fluid 28 may be maintained continuously to provide for clear viewing of the underlying tissue. Alternatively, the fluid 28 may be pumped temporarily or sporadically only until a clear view of the tissue is available to be imaged and recorded, at which point the fluid flow 28 may cease and blood 30 may be allowed to seep or flow back into imaging hood 12. This process may be repeated a number of times at the same tissue region or at multiple tissue regions.

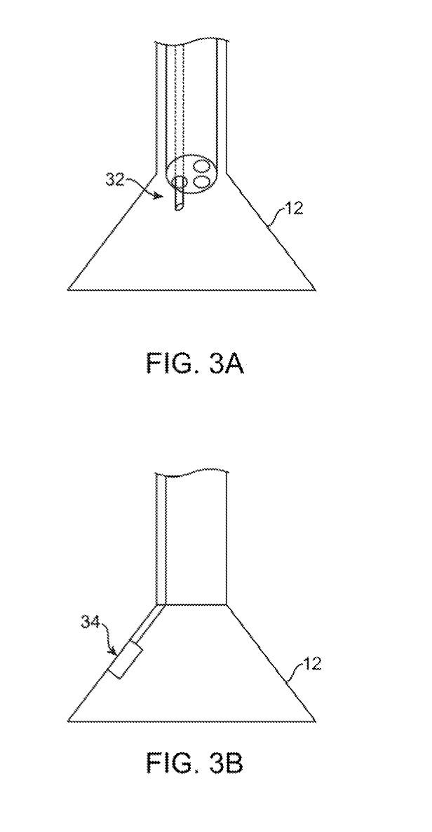

FIG. 3A shows a partial cross-sectional view of an example where one or more optical fiber bundles 32 may be positioned within the catheter and within imaging hood 12 to provide direct in-line imaging of the open area within hood 12. FIG. 3B shows another example where an imaging element 34 (e.g., CCD or CMOS electronic imager) may be placed along an interior surface of imaging hood 12 to provide imaging of the open area such that the imaging element 34 is off-axis relative to a longitudinal axis of the hood 12, as described in further detail below. The off-axis position of element 34 may provide for direct visualization and uninhibited access by instruments from the catheter to the underlying tissue during treatment.

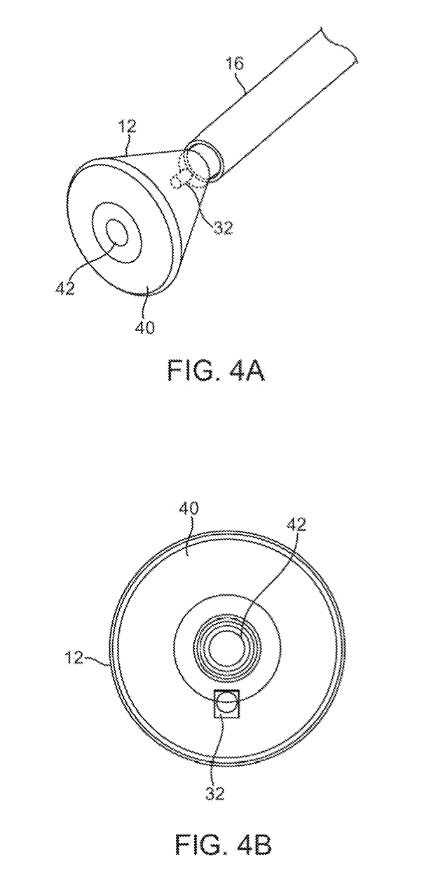

In utilizing the imaging hood 12 in any one of the procedures described herein, the hood 12 may have an open field which is uncovered and clear to provide direct tissue contact between the hood interior and the underlying tissue to effect any number of treatments upon the tissue, as described above. Yet in additional variations, imaging hood 12 may utilize other configurations. An additional variation of the imaging hood 12 is shown in the perspective and end views, respectively, of FIGS. 4A and 4B, where imaging hood 12 includes at least one layer of a transparent elastomeric membrane 40 over the distal opening of hood 12. An aperture 42 having a diameter which is less than a diameter of the outer lip of imaging hood 12 may be defined over the center of membrane 40 where a longitudinal axis of the hood intersects the membrane such that the interior of hood 12 remains open and in fluid communication with the environment external to hood 12. Furthermore, aperture 42 may be sized, e.g., between 1 to 2 mm or more in diameter and membrane 40 can be made from any number of transparent elastomers such as silicone, polyurethane, latex, etc. such that contacted tissue may also be visualized through membrane 40 as well as through aperture 42.

Aperture 42 may function generally as a restricting passageway to reduce the rate of fluid out-flow from the hood 12 when the interior of the hood 12 is infused with the clear fluid through which underlying tissue regions may be visualized. Aside from restricting out-flow of clear fluid from within hood 12, aperture 42 may also restrict external surrounding fluids from entering hood 12 too rapidly. The reduction in the rate of fluid out-flow from the hood and blood in-flow into the hood may improve visualization conditions as hood 12 may be more readily filled with transparent fluid rather than being filled by opaque blood which may obstruct direct visualization by the visualization instruments.

Moreover, aperture 42 may be aligned with catheter 16 such that any instruments (e.g., piercing instruments, guidewires, tissue engagers, etc.) that are advanced into the hood interior may directly access the underlying tissue uninhibited or unrestricted for treatment through aperture 42. In other variations wherein aperture 42 may not be aligned with catheter 16, instruments passed through catheter 16 may still access the underlying tissue by simply piercing through membrane 40.

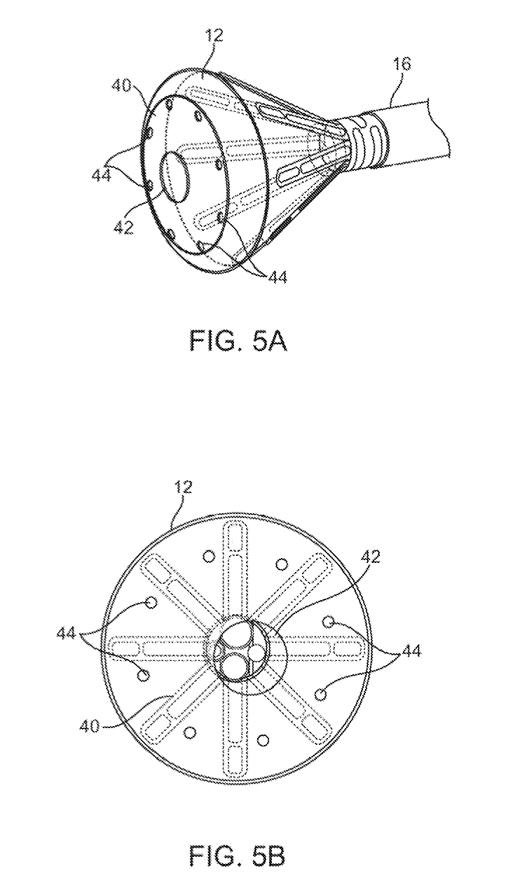

In an additional variation, FIGS. 5A and 5B show perspective and end views, respectively, of imaging hood 12 which includes membrane 40 with aperture 42 defined therethrough, as described above. This variation includes a plurality of additional openings 44 defined over membrane 40 surrounding aperture 42. Additional openings 44 may be uniformly sized, e.g., each less than 1 mm in diameter, to allow for the out-flow of the translucent fluid therethrough when in contact against the tissue surface. Moreover, although openings 44 are illustrated as uniform in size, the openings may be varied in size and their placement may also be non-uniform or random over membrane 40 rather than uniformly positioned about aperture 42 in FIG. 5B. Furthermore, there are eight openings 44 shown in the figures although fewer than eight or more than eight openings 44 may also be utilized over membrane 40.

Additional details of tissue imaging and manipulation systems and methods which may be utilized with apparatus and methods described herein are further described, for example, in U.S. patent application Ser. No. 11/259,498 filed Oct. 25, 2005 (U.S. Pat. Pub. 2006/0184048 A1), which is incorporated herein by reference in its entirety.

In utilizing the devices and methods above, various procedures may be accomplished. One example of such a procedure is crossing a tissue region such as in a transseptal procedure where a septal wall is pierced and traversed, e.g., crossing from a right atrial chamber to a left atrial chamber in a heart of a subject. Generally, in piercing and traversing a septal wall, the visualization and treatment devices described herein may be utilized for visualizing the tissue region to be pierced as well as monitoring the piercing and access through the tissue. Details of transseptal visualization catheters and methods for transseptal access which may be utilized with the apparatus and methods described herein are described in U.S. patent application Ser. No. 11/763,399 filed Jun. 14, 2007 (U.S. Pat. Pub. 2007/0293724 A1), which is incorporated herein by reference in its entirety. Additionally, details of tissue visualization and manipulation catheter which may be utilized with apparatus and methods described herein are described in U.S. patent application Ser. No. 11/259,498 filed Oct. 25, 2005 (U.S. Pat. Pub. 2006/0184048 A1), which is incorporated herein by reference in its entirety.

Generally, for the visualization and treatment devices to traverse through a punctured tissue wall, the opening through the tissue wall is typically dilated prior to passage. This may typically require several withdrawals and exchanges of various instruments such as a dilator or ablation device to widen the tissue opening to allow for the atraumatic passage of instruments such as the visualization and treatment device.

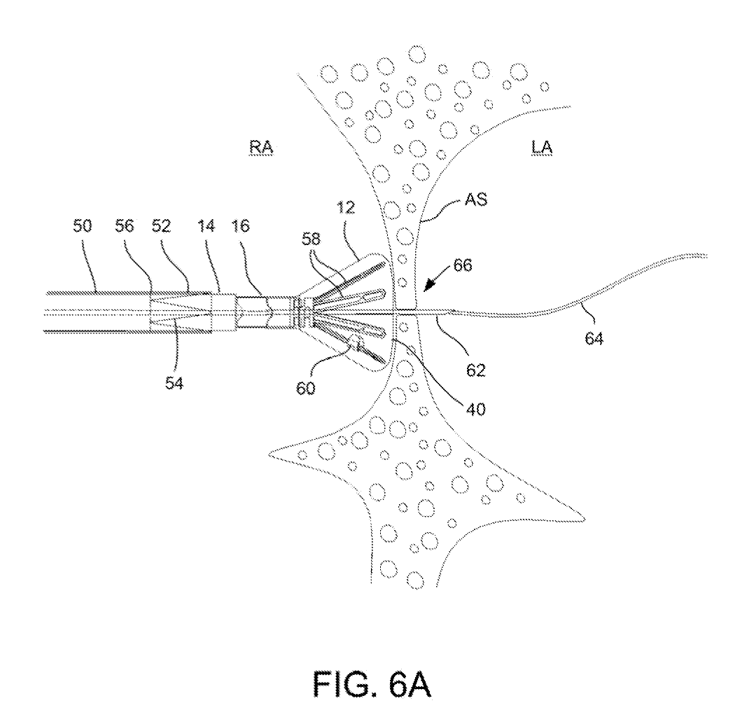

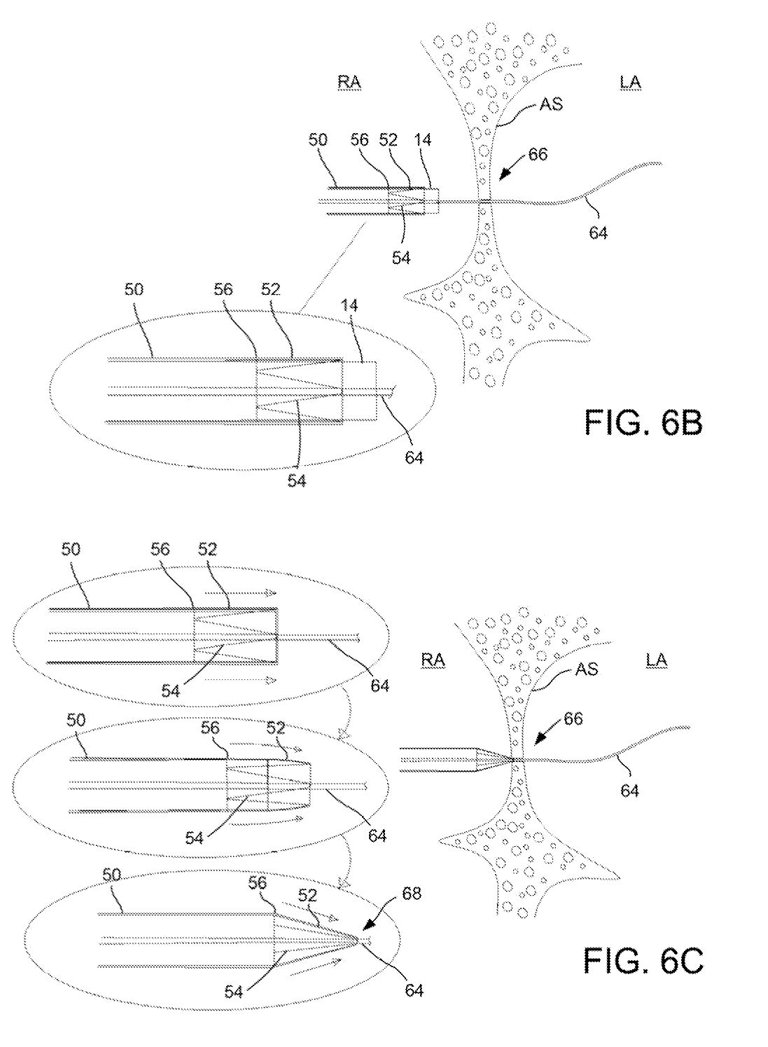

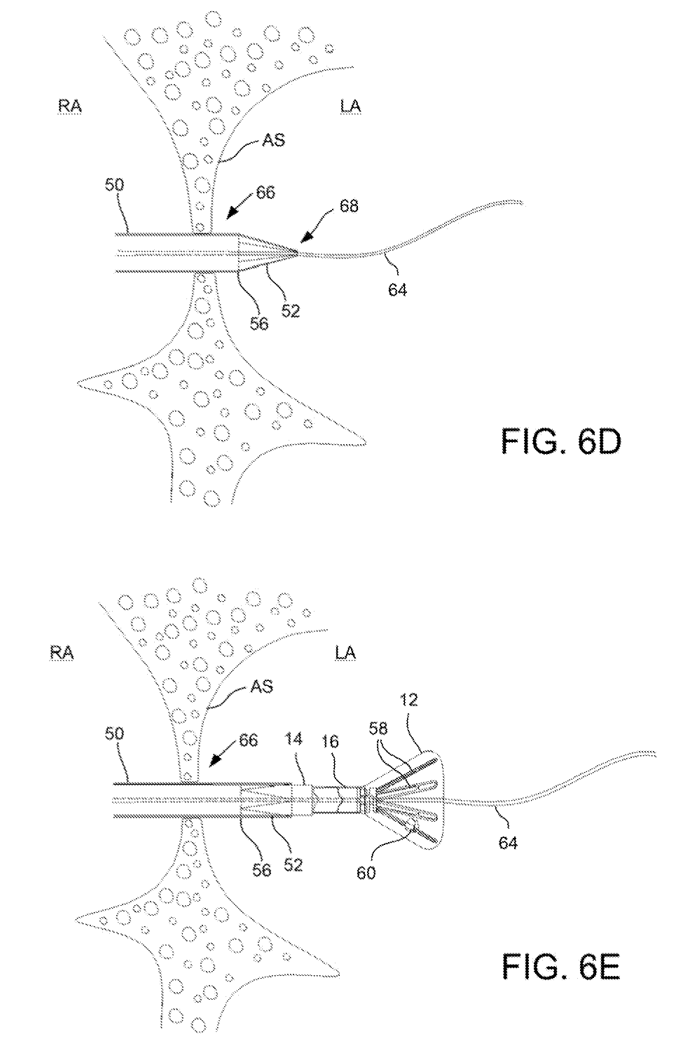

However, one example of a device which may allow for the penetration and rapid crossing of a tissue wall is illustrated in the partial cross-sectional side view of FIG. 6A, which shows a visualization device advanced through one example of a dilation sheath 50. Such a dilation sheath 50 may have a flexible length through which the visualization assembly may be advanced and a dilation assembly 52, which is shown in its opened configuration, positioned along a distal end of sheath 50 which may enable the penetration and rapid crossing of a septal wall without the use of a separate dilator and also without having to withdraw the visualization and treatment device from the patient body to allow for introduction of a separate dilator and the subsequent reinsertion of the visualization device.

As illustrated, hood 12 may project from deployment catheter 16 into its deployed configuration for positioning against the septal wall, which may be imaged to visually confirm a location of hood 12, e.g., along the fossa ovalis. Sheath 14 may be optionally deployed as well for conveying hood 12 and catheter 16. With hood 12 placed against the tissue surface and visual confirmation of the tissue location obtained, a piercing needle 62 may be advanced through catheter 16 and hood 12 to pierce into and through the atrial septum AS while under visualization from imager 60, which may be optionally positioned along one or more of the support struts 58 along hood 12. A guidewire 64 may be introduced through the needle 62 and also through the formed tissue opening 66 such that guidewire 64 passes from the right atrium RA to the left atrium LA. As previously mentioned, further examples for transseptal access procedures are described in U.S. patent application Ser. No. 11/763,399, which has been incorporated by reference.

With guidewire 64 passing through opening 66, the visualization assembly may be withdrawn directly within dilation sheath 50 or optionally within sheath 14, as shown in the cross-sectional view and detail view of FIG. 6B. As sheath 14, or the visualization device itself, is further withdrawn within dilation sheath 50 or as dilation sheath 50 is advanced over sheath 14 or the visualization device, the dilation assembly 52 may be biased to collapse or reconfigure itself into a tapered dilation configuration, as shown in cross-sectional and detail views of FIG. 6C. Dilation assembly 52 may be comprised, in one example, of a covering or extension which is formed of several triangular or saw-tooth shaped portions interconnected by an elastomeric substance in an alternating pattern along biased portions 54 and attached to dilation sheath at attachment 56. As the sheath 14 or visualization device is withdrawn and dilation assembly 52 is unconstrained, the elastomeric portions may be biased to draw each individual portion towards one another to collapse the assembly 52 while forming a guidewire opening 68 through which guidewire 64 may pass. Alternatively, dilation assembly 52 may be formed of a single construct such as a distensible membrane or covering which is biased to collapse when a constraint is removed.

In either case, once dilation assembly 52 has been reconfigured into its tapered configuration, dilation sheath 50 with visualization device positioned within may be advanced along guidewire 64 and through tissue opening 66 until dilation assembly 52 is positioned distal to opening 66 within, e.g., left atrium LA, as shown in FIG. 6D. The tapered configuration of dilation assembly 52 may accordingly facilitate the dilation and passage of the dilation sheath 50 through the atrial septum AS. Once dilation assembly 52 has passed desirably through the septal wall, sheath 14 (or the visualization device itself) may be advanced relative to dilation sheath 50 such that dilation assembly 52 is expanded back to its opened configuration to allow for catheter 16 and hood 12 to be deployed again while in the left atrium LA, as shown in FIG. 6E, where it may be advanced into proximity to any region of tissue for visualization and/or treatment such as the ostial tissue around the pulmonary veins, electrophysiological signal mapping, visualization, ablation, or other therapeutic and diagnostic procedures. Examples for use of the visualization catheter for ablation under direct visualization which may be utilized with the apparatus and methods described herein are described in further detail in U.S. patent application Ser. No. 11/775,819 filed Jul. 10, 2006 (U.S. Pat. Pub. 2008/0015569 A1), which is incorporated herein by reference in its entirety.

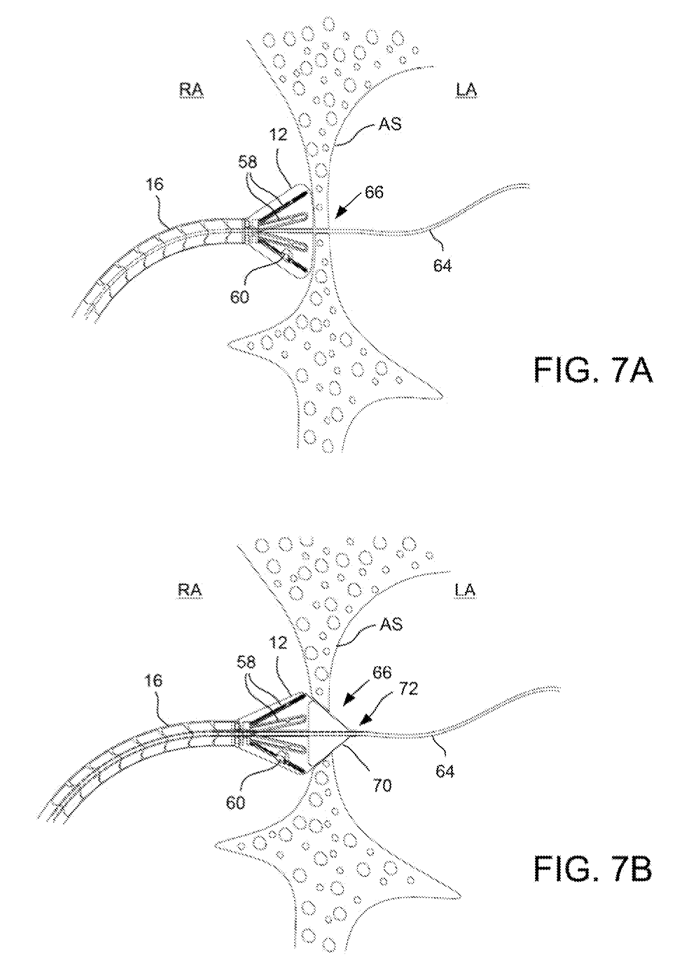

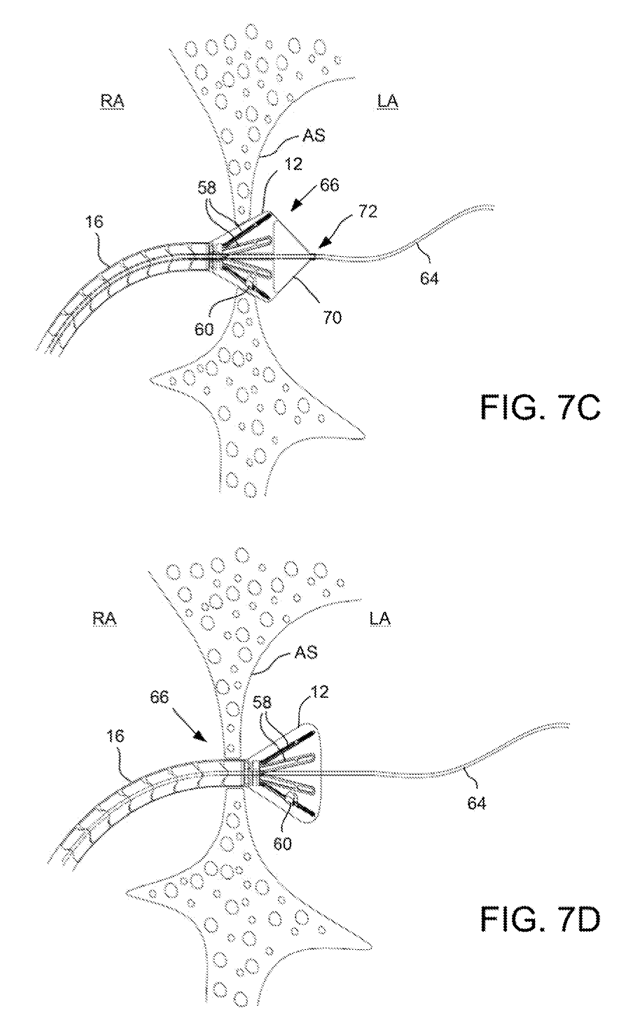

In yet another example, FIG. 7A illustrates a visualization catheter hood 12 similarly positioned against the atrial septum AS within the right atrium RA with guidewire 64 pierced through into the left atrium LA, as previously described. In this variation, hood 12 may incorporate an inflatable dilator balloon 70 which is optionally integrated along a distal end of hood 12 and expanded to form a conical dilator which projects distally of hood 12 while maintaining a guidewire opening 72. Alternatively, a deflated conically-shaped dilation balloon may be advanced from one of the working channels of the catheter 16 and inflated within the visualization hood 12 into a conical shape. In either variation, the inflated conical dilation balloon 70 may be subsequently deployed and pushed distally along the guidewire 64 to enlarge the transseptal puncture 66 to dilate the opening while hood 12 remains in its deployed configuration, as illustrated in FIG. 7B. Bio-compatible elastomeric balloon materials, such as Chronoflex™ or Chronoprene™, and which are optionally transparent may be utilized for fabricating the conical dilation balloon 70.

As shown in FIG. 7C, the conical dilation balloon 70 may be pushed further distally to allow the deployed hood 12 to be pushed across the septal wall AS. Upon the introduction of the conical dilation balloon 70 and hood 12 into the left atrium LA, the dilation balloon 70 may be deflated and/or retracted from hood 12, as shown in FIG. 7D. The hood 12 may then be articulated to the desired tissue region in the left atrium LA under the aid of direct visualization provided by hood 12 to perform any number of therapies such as ablation for atrial fibrillation or diagnostics such as electrophysiological mapping and pacing, etc.

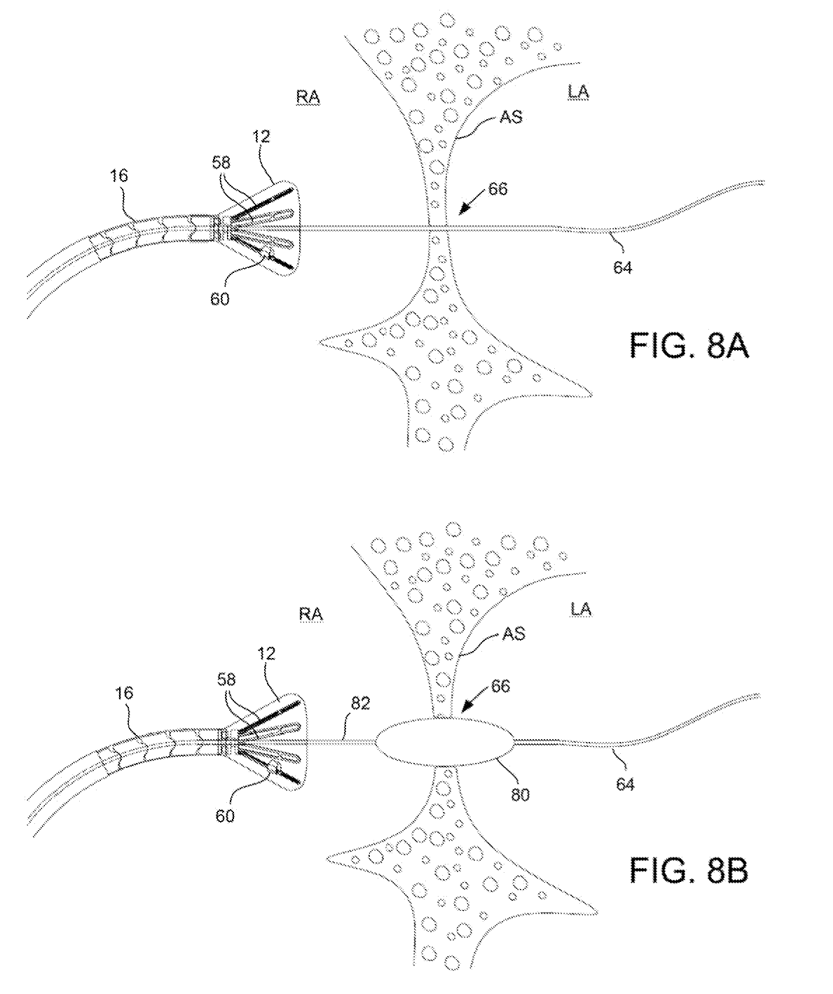

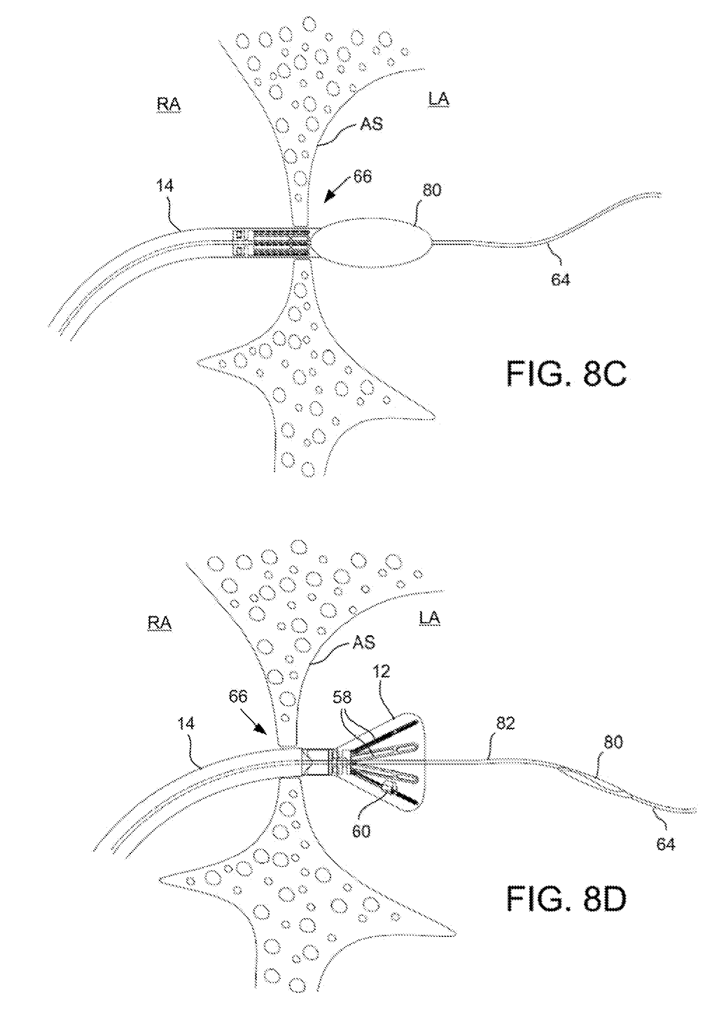

In yet another variation, the visualization device may be utilized with an inflatable balloon dilator. After introduction of the guidewire 64 into the left atrium LA, as shown in FIG. 8A, a dilation balloon shaft 82 having a dilation balloon 80 may be advanced over the guidewire 64 in an uninflated state and into the undilated tissue opening 66. When desirably positioned within opening 66, dilation balloon 80 may be inflated to expand the opening 66 to a diameter which is at least as wide as catheter 16 if not wider, as shown in FIG. 8B, or optionally as wide as deployed hood 12.

While maintaining dilation balloon 80 in its inflated state, hood 12 may be collapsed and withdrawn within sheath 14 and the sheath 14, with the retracted hood 12 positioned within its lumen, may then be advanced relative to balloon 80 so that the distal opening of sheath 14 is just proximal to inflated balloon 80. In this manner, both balloon 80 and sheath 14 may be pushed through the atrial septum AS and into the left atrium LA, as illustrated in FIG. 8C, until both the balloon 80 and the distal opening of sheath 14 has cleared tissue opening 66. By maintaining the close proximity between balloon 80 and sheath 14, the sheath 14 may follow through the tissue wall uninhibited by the transition between the balloon 80 and sheath 14. Once the sheath 14 with the collapsed hood 12 has gained access into the left atrium LA, hood 12 may be readily redeployed from sheath 14, as shown in FIG. 8D, and the dilation balloon 80 may be deflated and subsequently withdrawn through hood 12 and catheter 16.

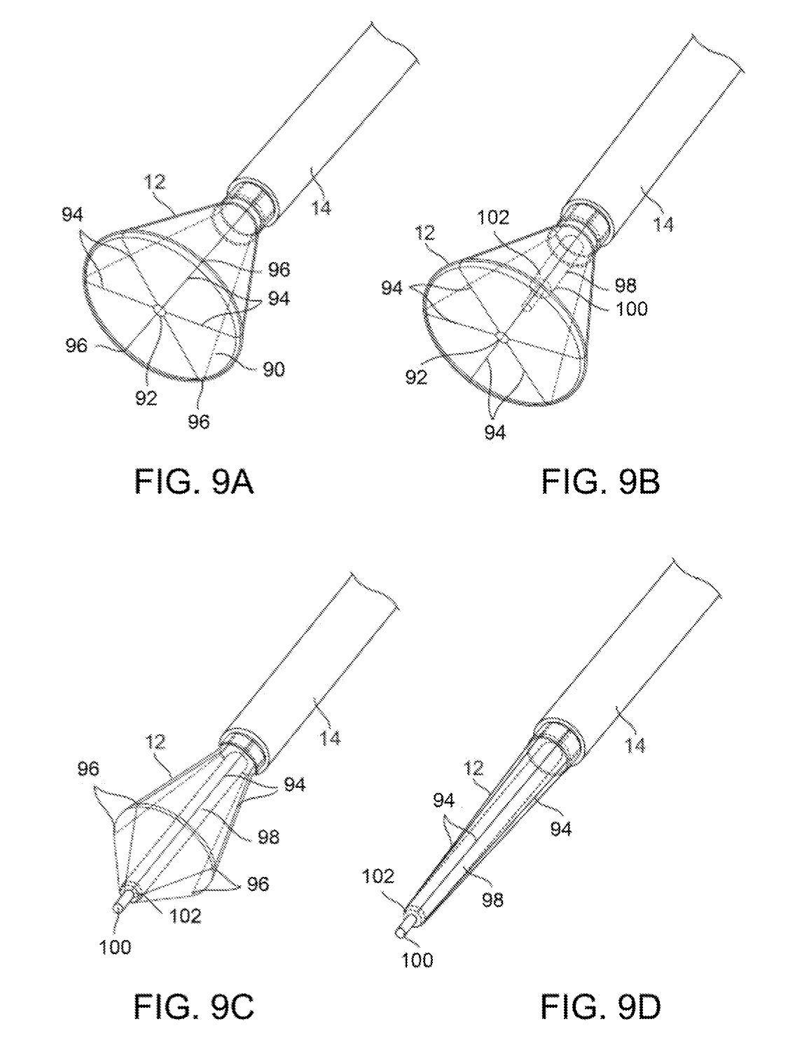

FIGS. 9A to 9D illustrate perspective views of yet another variation of a hood assembly covered by a membrane 90 and which defines an aperture 92 having a diameter of, e.g., 1 to 4 mm, over membrane 90 at a distal end of hood 12. This variation in particular shows an example of an assembly which is configured to restrict or control fluid flow into and out of hood 12 and which is also collapsible into a low-profile configuration which is utilizable as a tissue dilator. Features of this particular variation are shown and described in further detail in U.S. patent application Ser. No. 12/026,455 filed Feb. 5, 2008 (U.S. Pat. Pub. 2008/0188759 A1), which is incorporated herein by reference in its entirety.

As shown, hood 12 may be defined by several support struts 94 made from materials such as Nitinol, nylon, Mylar, etc., which extend from the proximal end of hood 12 and define curved or bent portions 96 which terminate at the distal end of hood 12 at the flow control aperture 92. A strut may also form a ring surrounding aperture 92 to provide circumferential strength to aperture 92, as shown in FIG. 9A. In its deployed configuration, hood 12 with aperture 92 may be utilized to visualize and/or treat tissue while restricting or controlling the flow of fluid from and into hood 12 via aperture 92. To deploy and/or collapse hood 12 between its deployed and low-profile configurations, an instrument 98 such as a dilator having an atraumatic tip 100 projecting distally from a shoulder 102 may be advanced distally through the deployment catheter and into hood 12, as shown in FIG. 9B. In particular, atraumatic tip 100 may be electrically coupled to a power source, such as an RF generator, such that tip 100 is energizable with RF energy and functions as a bipolar or monopolar energizable cutting electrode. Accordingly, tip 100 may be fabricated from an electrically conductive material such as platinum, gold, stainless steel, Nitinol, etc. Tip 100 may be alternatively energized via other forms of energy such as laser energy, cryo-energy, etc.

Instrument 98 may be further advanced until tip 100 projects through aperture 92 and shoulder 102 engages or abuts against the interior of membrane 90 surrounding aperture 92. As instrument 98 is pushed further distally, the curved or bent portions 96 of support struts 94 may become start to become straightened relative to instrument 98 and support struts 94 may begin to collapse, as shown in FIG. 9C. Once instrument 98 has been fully advanced into its distal position, portions 96 and support struts 94 may be fully collapsed against instrument 98 into a low-profile configuration, as shown in FIG. 9D. In addition to tip 100, all or selected numbers of support struts 94 may be coupled to a power source, such as the same RF generator coupled to tip 100, and may have exposed portions which are energizable to facilitate dilation or cutting of the tissue opening 66 to facilitate the passage of the collapsed hood 12.

With this variation, hood 12 may be collapsed for delivery without having to retract hood 12 into a catheter sheath 14. Additionally, with the ability to collapse hood 12 distally rather than proximally, projecting tip 100 may be used to cut into or through tissue via its energized tip and to also actively dilate tissue openings, cavities, flaps, etc. such as the fossa ovalis or the coronary sinus. With direct dilation, hood 12 may be guided to pass through the tissue opening, cavity, or flap in a single process. Procedures such as transseptal access or coronary sinus cannulation can therefore be performed more efficiently.

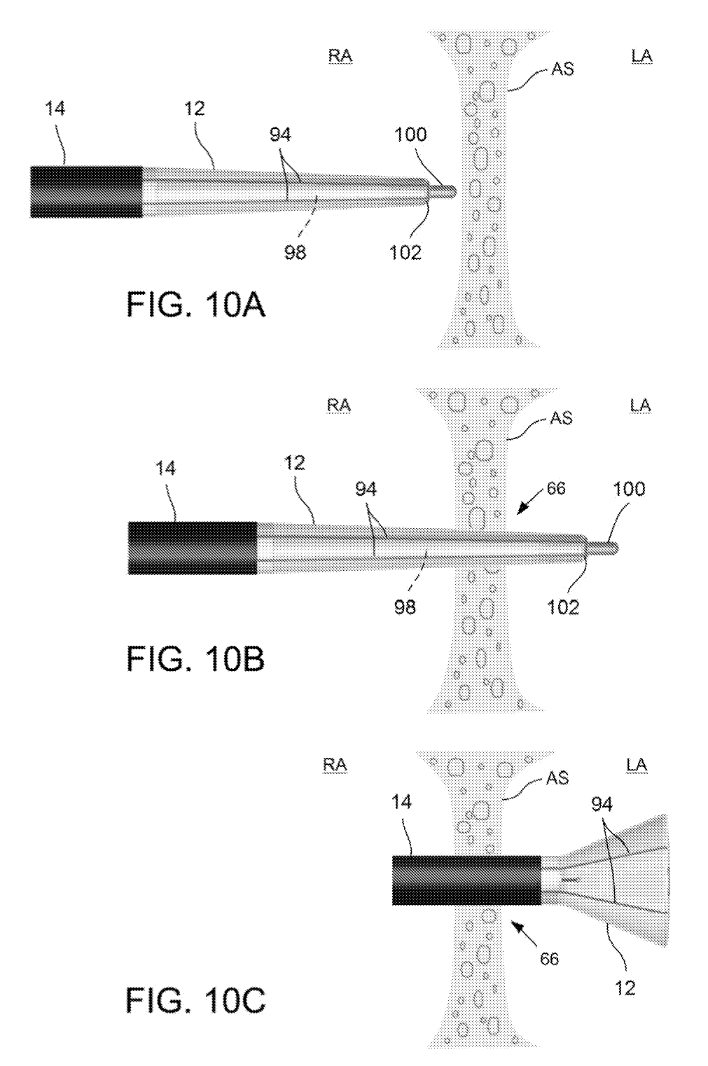

FIGS. 10A to 10C illustrate an example of the collapsed hood 12 advanced through an atrial septum AS utilizing energized tip 100. With hood 12 in its low-profile configuration with tip 100 extended distally, RF energy may be applied to tip 100 when in contact with or in proximity to the tissue region to be crossed, as shown in FIG. 10A. As the energized tip 100 begins to cut through the tissue, hood 12 may be urged distally through tissue opening 66. One or more struts 94 may be optionally energized to facilitate the cutting and dilation of the opening 66 as hood 12 is urged further through the opening 66. Once the streamlined low-profile hood 12 is fully positioned within the left atrium LA, the tip 100 may then be withdrawn proximally to restore hood 12 back into its deployed configuration, as shown in FIG. 10C.

Methods and apparatus disclosed herein may also be used with visualization and ablation catheters, such as steerable visual electrode ablation catheters, for rapid transseptal access to the left atrium LA of the heart. Details of such devices and methods which may be utilized herewith are described in further detail in U.S. patent application Ser. No. 12/118,439 filed May 9, 2007 (U.S. Pat. Pub. 2009/0030412 A1), which is incorporated herein by reference in its entirety.

The applications of the disclosed invention discussed above are not limited to certain treatments or regions of the body, but may include any number of other treatments and areas of the body. Modification of the above-described methods and devices for carrying out the invention, and variations of aspects of the invention that are obvious to those of skill in the arts are intended to be within the scope of this disclosure. Moreover, various combinations of aspects between examples are also contemplated and are considered to be within the scope of this disclosure as well.

What is claimed is:

1. A tissue dilation instrument, comprising:
a flexible elongate shaft defining a lumen therethrough;
a dilation assembly which is attached to a distal end of the shaft, and
wherein the assembly is reconfigurable between an open configuration which allows communication with the lumen and a tapered configuration when unconstrained.

2. The instrument of claim 1 wherein the dilation assembly is biased to collapse from the open configuration to the tapered configuration.

3. The instrument of claim 1 wherein the dilation assembly is comprised of a plurality of triangular portions connected to one another in an alternating pattern.

4. The instrument of claim 1 wherein the dilation assembly is comprised of a biased distensible membrane.

5. The instrument of claim 1 wherein the dilation assembly tapered configuration defines an opening therethrough.

6. The instrument of claim 1 further comprising a visualization device having a reconfigurable hood which is positionable within the lumen of the elongate shaft.

7. A method of dilating a tissue region, comprising:
retracting a device within a lumen relative to a dilation assembly attached to a distal end of a flexible elongate shaft such that the dilation assembly reconfigures from an open configuration to a tapered configuration; and
advancing the elongate shaft and the dilation assembly in the tapered configuration into an opening in the tissue region such that the opening is dilated by the dilation assembly.

8. The method of claim 7 further comprising introducing a guidewire through the opening in the tissue region prior to advancing the elongate shaft.

9. The method of claim 8 wherein introducing further comprises visualizing the tissue region.

10. The method of claim 7 wherein advancing the elongate shaft comprises advancing the shaft along a guidewire passing through the opening.

11. The method of claim 7 wherein advancing the elongate shaft comprises passing the dilation assembly through an atrial septum from a right atrium to a left atrium of a heart.

12. The method of claim 7 further comprising passing the dilation assembly through the opening and into a left atrium of a heart.

13. The method of claim 12 further comprising advancing the device within the lumen and through the dilation assembly such that the assembly reconfigures from the tapered configuration to the open assembly.

14. The method of claim 7 wherein retracting a device comprises retracting a visualization assembly having an expandable hood.

15. A tissue dilation instrument, comprising:
a flexible elongate shaft having a hood projecting distally therefrom which is reconfigurable between a low profile and an expanded profile; and
an inflatable dilation balloon having a conical shape extending distally from the hood when in the expanded profile.

16. The instrument of claim 15 further comprising an imager positioned within or along the hood.

17. The instrument of claim 15 wherein the dilation balloon is integrated with the hood.

18. The instrument of claim 15 wherein the dilation balloon is separable from the hood.

19. A tissue dilation system, comprising:
a flexible elongate shaft having a hood projecting distally therefrom which is reconfigurable between a low profile and an expanded profile; and
an inflatable dilation balloon extending from a balloon shaft which is translatable through the elongate shaft and the hood.

20. The system of claim 19 further comprising an imager positioned within or along the hood.

21. The system of claim 19 wherein the dilation balloon has an inflation diameter which is at least as wide as a diameter of the elongate shaft.

22. The system of claim 19 wherein the elongate shaft is positionable proximally of the dilation balloon in an inflated configuration when the hood is in its low profile.
